(19) United States
(12) Patent Application Publication
(10) Pub. No.: US 2024/0148939 A1
(43) Pub. Date: May 9, 2024

(54) BIPHASIC BIOMATERIAL BASED ON CURDLAN AND HYDROXY APATITE (HAP) FOR REGENERATION OF OSTEOCHONDRAL DEFECTS AND THE METHOD OF ITS PREPARATION

(71) Applicant: UNIWERSYTET MEDYCZNY W LUBLINIE, Lublin (PL)

(72) Inventors: Katarzyna KLIMEK, Lublin (PL); Grazyna GINALSKA, Lublin (PL); Marta TARCZYNSKA-OSINIAK, Lublin (PL)

(21) Appl. No.: 18/281,286

(22) PCT Filed: Mar. 7, 2022
(86) PCT No.: PCT/IB2022/052002

§ 371 Date: Sep. 8, 2023

(30) Foreign Application Priority Data
Mar. 8, 2021 (PL) P.437234

(51) Int. Cl. A61L 27/42; A61L 27/22

(52) U.S. Cl. CPC A61L 27/425; A61L 27/227; A61L 2400/12; A61L 2430/02

(57) ABSTRACT

The curdlan-based biomaterial containing P-1,3-glucan (curdlan), whey protein isolate (WPI) and hydroxyapatite ceramics (HAp), where the proportions of polymer components to 100 ml of aqueous solution are respectively: 6-20% (w/v)—P-1,3-glucan, 20-50% (w/v)—whey protein isolate (WPI), while the amount of added hydroxyapatite ceramic granules (HAp) to such a polymer mixture is 40 g-100 g. and a method of producing the biphasic biomaterial consisting in that a 20-50% (w/v) aqueous solution of whey protein isolate (WPI), preferably 30% (w/v), is added to the P-1,3-glucan powder (curdlan), so as to obtain a mixture in which the concentration of curdlan in relation to the WPI solution is 6-20% (w/v), preferably 8% (w/v).

The subject of the invention is a biomimetic biphasic biomaterial based on the natural polysaccharide β-1,3-glucan (curdlan), containing two coherent phases: the upper one—polymeric, consisting of curdlan and whey protein isolate (WPI), and the lower one—polymer-ceramic containing curdlan, WPI, and hydroxyapatite (HAp). The biomaterial could be used in regeneration of osteochondral defects. The aspect of the invention is also the method of its preparation.

The osseochondral lesions (Grade IV according to the Outerbridgae classification) are the most serious and, at the same time, the most common problems among orthopedic patients. They include not only the cartilage, but also the subchondral bone (Casey Slattery B. S., Clin. Orthop. Relat. Res. 2018, 476, 2101-2104; Cassar-Gheiti A. J. et al. Intechopen 2016, doi: 10.5772/intechopen.70261). Cartilage differs significantly from bone tissue both in composition and in structural, mechanical and biological properties. Moreover, cartilage, unlike bones, has little regenerative capacity. Therefore, the use of homogeneous biomaterials may not be sufficient. Repair of such complex lesions with the use of phase/layered biomaterials that mimic both cartilage and bone tissue currently gives the best therapeutic results (Deng C. et al. Nanomed. Nanobiotechnol. 2019, e1576; Guarnino V. et al. Materials for Biomedical Engineering, Chapter 10, 2019, 297-337; Li X. et al. Regen. Biomater. 2015, 2, 221-228).

A known solution in the osseochondral lesions repair is the use of biomimetic phase/layered biomaterials consisting of natural and/or synthetic polymers and inorganic components, e.g. calcium phosphate ceramics, bioglasses etc. For example, from the patent specification no. CN106178126A, there is known a biphasic biomaterial consisting of an upper phase containing silk fibroin and chitosan (SF-CS) and a lower phase additionally enriched with nanohydroxyapatite (SF-CS-nHA). In vivo studies show that the biomaterial prepared according to the invention has regenerative properties for cartilage and bone tissue (studies on New Zealand rabbits) and therefore it can be used in osseochondral lesions repair. From a patent description no. WO2017118863A1 there is known a biomimetic biphasic biomaterial that contains collagen and a copolymer of lactic and glycolic acid (PLGA) (upper phase), and collagen combined with hydroxyapatite (HAp), deposited on a titanium substrate (lower phase). The biomaterial has mechanical properties similar to that of native cartilage and bone tissue, and promotes cell growth and proliferation over time (in vitro studies using stem cells derived from sheep bone marrow). In addition, in vivo studies confirmed the integration of the scaffold with the cartilage and bone of sheep and Spinone Italiano, which indicates that the biomaterial can be used in the regeneration of osseochondral lesions. From the patent description no. US2015110846A1 there is known a two-phase biomaterial based on nanofibers composed of chondroitin sulfate and hyaluronic acid (upper phase) and nanofibers containing hydroxyapatite, β-glycerophosphate and β-TCP (lower phase). The biomaterial promotes the colonization and proliferation of human chondrocytes in vitro. In turn, from the patent description no. WO2010084481A1, a three-layer biomaterial for the repair of osseochondral lesions is known. The upper layer of the biomaterial consists of collagen type I and collagen type 11 (1:1), the intermediate layer consists of collagen type I, collagen type II (1:1) and hydroxyapatite, and the lower layer consists only of hydroxyapatite. This scaffold supports the proliferation of normal mouse pre-osteoblasts in vitro (MC3T3-E1 cell line).

The review of the patent databases and the above patents shows that there are currently no phase/layered biomaterials containing bacterial β-1,3-glucan (curdlan), whey protein isolate (WPI) and hydroxyapatite ceramics. However, it is known that the β-1,3-glucan (curdlan) hydrogel obtained by the thermal method has been applied as a component of a bone substitute based on hydroxyapatite ceramics (Polish Patent no. PL 206394 and International Patent no. EU 2421570 B1). In turn, the curdlan hydrogel obtained by dialysis against a solution of calcium ions has been used as a component of a biocompatible ceramic-polymer bone scaffold (Polish Patent PL 229329 B1). In addition, there is known a hydrogel based on whey protein isolate (WPI), as well as hydrogels based on WPI and polysaccharides amylose, cellulose, amylopectin, chitosan and dextran, which can be used in the bone tissue repair (US Patent US/2013/0101548 A1), U.S. Pat. No. 9,758,558 B2 and International Patent No. WO 2011/123760 A2). It is worth noting, however, that the aforementioned inventions are composite biomaterials in which the components are uniformly distributed to each other—there are no visible phases in their structure.

The presented invention solves the problem of obtaining a biphasic biomaterial, consisting of: the upper—polymer phase, comprising β-1,3-glucan (curdlan) and whey protein isolate (WPI), and the lower—polymer-ceramic phase, comprising curdlan, WPI and hydroxyapatite (HAp), characterized by the absence of cytotoxicity and biocompatibility in vitro by promoting the growth and proliferation of human osteoblasts (hFOB 1.19 line, ATCC).

The biphasic biomaterial according to the invention is composed of β-1,3-glucan (curdlan), whey protein isolate (WPI) and hydroxyapatite ceramics (HAp), where the proportions of polymer components to 100 ml of aqueous solution are, respectively: 6-20% (w/v)—β-1,3-glucan, 20-50% (w/v)—whey protein isolate (WPI), while the amount of added hydroxyapatite ceramic granules (HAp) to such a polymer mixture is 40 g-100 g, whereby the upper phase is a mixture of β-1,3-glucan (curdlan) and whey protein isolate (WPI), while the lower phase is a mixture of β-1,3-glucan (curdlan) and whey protein isolate (WPI) and hydroxyapatite ceramics (HAp).

The biomaterial preferably contains 8% (w/v) β-1,3-glucan related to 100 ml of the aqueous solution.

The biomaterial preferably contains whey protein isolate (WPI) in an amount of 30% (w/v) related to 100 ml of the aqueous solution.

Preferably, the hydroxyapatite ceramic (HAp) is in the form of a nanopowder or powder or granules of 0.01-1.0 mm size.

The biomaterial preferably contains a hydroxyapatite ceramic (HAp) in an amount of 70-80 g related to 100 ml of aqueous polymer solution.

The invention also concerns a β-1,3-glucan (curdlan) and hydroxyapatite ceramic (HAp)-based biphasic biomaterial described above for application in osseochondral lesions repair.

A method for the production of a biphasic curdlan-based biomaterial for the regeneration of osseochondral lesions, characterized in that 20-50% (w/v) aqueous solution of whey protein isolate (WPI), preferably 30% (w/v), is added to the β-1,3-glucan (curdlan), so that the concentration of curdlan in relation to the WPI solution is 6-20% (w/v), preferably 8% (w/v), then hydroxyapatite ceramic (HAp) in the form of a nanopowder, powder or granules of 0.01-1.0 mm size is added to this mixture, sintered at the temperature of 500-1300° C., in the amount of 40-100 g per 100 ml of aqueous polymer solution, then the obtained mixture is subjected to centrifugation for 1-10 minutes, at a speed of 800-10,000 rpm, then the mixture with the formed upper—polymeric and the lower—polymer-ceramic phases is incubated preferably for 15 minutes at a temperature of 90-120° C. and sterilized.

The method according to claim 2 characterized in that the granules are used in an amount of 40 to 100 g, most preferably 70-80 g (related to 100 ml of the polymer solution), sintered at a temperature of 1100-1200° C.

Preferably, sterilization is carried out by autoclaving in a wet form.

Preferably, sterilization of the wet material is carried out at 121° C. for 15 minutes.

Preferably, the biphasic biomaterial is dried at room temperature prior to gas sterilization.

Preferably, gas sterilization of the dry biomaterial is performed with ethylene oxide (55° C.) for approximately 3 hours.

Preferably the size of the granules is 0.05-0.2 mm.

Preferably, the resulting mixture is centrifuged for 3 minutes at 3000 rpm.

Preferably, the mixture with the formed two phases (upper—polymer and lower—polymer-ceramic) is incubated at 90° C.

The biphasic curdlan-based biomaterial produced according to the invention is characterized by favorable biological properties in relation to in vitro human osteoblast line hFOB 1.19.

The combination of polymers, i.e., β-1,3-glucan (curdlan) and whey protein isolate (WPI) as well as hydroxyapatite ceramic (HAp) in appropriate proportions and the method of combining them leading to a biphasic biomaterial, gave the desired effects in the osseochondral lesions repair.

As it has been unexpectedly found, these components complement each other, creating a biocompatible, coherent biomaterial, having two phases (polymer and polymer-ceramic), characterized by no delamination at their boundary. The non-toxic β-1,3-glucan (curdlan) in combination with biocompatible whey protein isolate (WPI) forms an elastic gel that allows the deposition of hydroxyapatite ceramics (HAp) in its lower part, which in turn allows obtaining a biomaterial consisting of two phases—the upper polymer (curdlan-WPI) and lower polymer-ceramic (curdlan-WPI-HAp). Both phases of the biomaterial show desirable biological properties against human osteoblast cells by promoting their growth and proliferation in vitro, which indicates that the biphasic biomaterial can be used to repair osteochondral lesions. Until now, no one in the prior art has disclosed a biphasic biomaterial with the composition according to the invention.

The biomaterial is also characterized by biomimeticity, i.e., its structure and composition mimic the natural cartilage and bone.

Advantageous biological and structural properties of the biomaterial, i.e., the ability to support the viability and proliferation of hFOB 1.19 osteoblasts as well as cohesiveness and biomimeticity result from its qualitative and quantitative composition and the method of its production.

The two-phase nature of the material according to the invention is achieved by the use of certain components and their amount, especially ceramics (HAp) in an amount of 40-100g with respect to the remaining components, and the production method, in particular by the use of centrifugation.

The invention is illustrated by the following examples.

### EXAMPLE 1

First, 1 ml of distilled water was added to 0.4 g of whey protein isolate (WPI). The ingredients were mixed until the WPI was completely dissolved. The obtained clear WPI solution was added to 0.08 g of curdlan placed in a 2 ml Eppendorf tube. The ingredients were mixed until they were completely combined. Then, 0.9 g of hydroxyapatite nanopowder was added. The resulting mixture was centrifuged for 1 minute at 9000 rpm, and then incubated at 90° C. for 15 minutes. The obtained biphasic biomaterial was sterilized by autoclaving (121° C., 15 minutes). The biomaterial prepared in this way has favorable properties, i.e., it does not show cytotoxicity and supports the proliferation of osteoblasts.

The obtained material had a biphasic structure, where the upper polymer phase constituted approx. 10% of the biomaterial volume, and the lower polymer-ceramic phase constituted approx. 90% of the biomaterial volume.

### EXAMPLE 2

First, 1 ml of distilled water was added to 0.3 g of whey protein isolate (WPI). The ingredients were mixed until the WPI was completely dissolved. The obtained clear WPI solution was added to 0.07 g of curdlan placed in a 2 ml Eppendorf tube. The ingredients were mixed until they were completely combined. Then, 1.0 g of hydroxyapatite powder was added. The resulting mixture was centrifuged for 9 minutes at 900 rpm, and then incubated at 90° C. for 15 minutes. The obtained biphasic biomaterial was sterilized by autoclaving (121° C., 15 minutes). The biomaterial prepared in this way has favorable properties, i.e., it does not show cytotoxicity and supports the proliferation of osteoblasts.

The obtained material had a biphasic structure, where the upper polymer phase constituted approx. 5% of the biomaterial volume, and the lower polymer-ceramic phase constituted approx. 95% of the biomaterial volume.

### EXAMPLE 3

First, 1 ml of distilled water was added to 0.3 g of whey protein isolate (WPI). The ingredients were mixed until the WPI was completely dissolved. The obtained clear WPI solution was added to 0.08 g of curdlan placed in a 2 ml Eppendorf tube. The ingredients were mixed until they were completely combined. Then, 0.7 g of hydroxyapatite granules, 0.05-0.2 mm in size, sintered at 1150° C., were added. The resulting mixture was centrifuged for 3 minutes at 3000 rpm, and then incubated at 90° C. for 15 minutes. The resulting biphasic biomaterial was removed from the tube and dried at room temperature for 24 hours. The sterilization of the obtained sample was carried out with the use of ethylene oxide (55° C., 3 hours, followed by a 15-hour ventilation of the sample in order to remove the residual ethylene oxide after the sterilization process). The biomaterial prepared in this way has favorable properties, i.e., it does not show cytotoxicity and supports the proliferation of osteoblasts.

The obtained material had a biphasic structure, where the upper polymer phase constituted approx. 30% of the biomaterial volume, and the lower polymer-ceramic phase constituted approx. 70% of the biomaterial volume.

The biphasic curdlan-based biomaterial, produced according to the invention, containing 0.08 g of curdlan, 0.3 g of whey protein isolate (WPI) and 0.05-0.2 mm of hydroxyapatite in the form of granules with a size of 0.05-0.2 mm in the amount of 0,7 g, is shown in the photo.

### EXAMPLE 4

First, 1 ml of distilled water was added to 0.35 g of whey protein isolate (WPI). The ingredients were mixed until the WPI was completely dissolved. The obtained clear WPI solution was added to 0.08 g of curdlan placed in a 2 ml Eppendorf tube. The ingredients were mixed until they were completely combined. Next, 0.5 g of hydroxyapatite granules, 0.3-0.6 mm in size, sintered at 1200° C., were added. The resulting mixture was centrifuged for 5 minutes at 3500 rpm, and then incubated at 90° C. for 15 minutes. The obtained biphasic biomaterial was removed from the tube and dried at room temperature for 24 hours. The sterilization of the obtained sample was carried out with the use of ethylene oxide (55° C., 3 hours, followed by a 15-hour ventilation of the sample in order to remove the residual ethylene oxide after the sterilization process). The biomaterial prepared in this way has favorable properties, i.e., it does not show cytotoxicity and supports the proliferation of osteoblasts.

### EXAMPLE 5

First, 1 ml of distilled water was added to 0.4 g of whey protein isolate (WPI). The ingredients were mixed until the WPI was completely dissolved. The obtained clear WPI solution was added to 0.08 g of curdlan placed in a 2 ml Eppendorf tube. The ingredients were mixed until they were completely combined. Then, 0.4 g of hydroxyapatite granules, 0.3-0.6 mm in size, sintered at 1150° C., were added. The resulting mixture was centrifuged for 6 minutes at 2000 rpm, and then incubated at 90° C. for 15 minutes. The obtained biphasic biomaterial was sterilized by autoclaving (121° C., 15 minutes). The biomaterial prepared in this way has favorable properties, i.e., it does not show cytotoxicity and supports the proliferation of osteoblasts.

The obtained material had a biphasic structure, where the upper polymer phase constituted approx. 60% of the biomaterial volume, and the lower polymer-ceramic phase constituted approx. 40% of the biomaterial volume.

### EXAMPLE 6

First, 1 ml of distilled water was added to 0.2 g of whey protein isolate (WPI). The ingredients were mixed until the WPI was completely dissolved. The obtained clear WPI solution was added to 0.15 g of curdane placed in 2 ml Eppendorf tubes. The ingredients were mixed until they were completely combined. Then, 0.7 g of hydroxyapatite granules, 0.05-0.2 mm in size, sintered at 1150° C., were added. The resulting mixture was centrifuged for 4 minutes at 4000 rpm, and then incubated at 90° C. for 15 minutes. The obtained biphasic biomaterial was sterilized by autoclaving (121° C., 15 minutes). The biomaterial prepared in this way shows favorable properties, i.e., it does not show cytotoxicity and supports the proliferation of osteoblasts. The obtained material had a biphasic structure, where the upper polymer phase constituted approx. 30% of the biomaterial volume, and the lower polymer-ceramic phase constituted approx. 70% of the biomaterial volume.

In order to assess the biological properties of the formed phases of the biomaterial produced according to the invention, i.e., the polymer phase and the polymer-ceramic phase, the scaffold was cut into discs. As a result, curdlan-WPI samples were obtained from the upper phase of the biomaterial—the polymer phase, imitating cartilage, and curdlan-WPI-HAp from the lower phase of the biomaterial—the polymer-ceramic phase, imitating subchondral bone.

Test results for the biomaterial curdlan (8% w/v)—WPI (30% w/v) and curdlan (8% w/v)—WPI (30% w/v)—HAp granules with a size of 0.05-0, 2 mm, sintered at 1150° C. (70 g of HAp granules per 100 ml of curdlan-WPI solution) prepared according to the invention, are shown in Table 1.

TABLE 1
Selected properties of the curdlan-WPI/curdlan-WPI-HAp biphasic biomaterial
prepared according to the invention, described in Example 3.

| | Curdlan - WPI/curdlan - WPI - HAp biomaterial | |
|---|---|---|
| | Curdlan-WPI samples, from the | Curdlan-WPI-HAp samples from the |
| Feature | upper phase of the biomaterial | lower phase of the biomaterial |
| The influence on the viability of hFOB | Supporting the viability of osteoblast | Supporting the viability of osteoblast |
| 1.19 osteoblasts (human osteoblasts | cells | cells |
| obtained from ATCC) - test using fluid | Viability was 102.86 ± 3.48% | Viability was 112.79 ± 5.05% |
| extracts. | compared to control cells. | compared to control cells. |
| The results were obtained in an indirect | | |
| test using fluid extracts obtained from | | |
| biomaterials, in accordance with the ISO | | |
| 10993-5 standard: Biological evaluation | | |
| of medical devices - Part 5: Tests for in | | |
| vitro cytotoxicity. Control cells were | | |
| exposed to the culture medium incubated | | |
| without the tested biomaterials. Results | | |
| are presented as mean values ± standard | | |
| deviation after 24-hour incubation of | | |
| cells with extracts (MTT test). | | |
| Proliferation of human hFOB 1.19 | Microscopic observations showed | Microscopic observations showed |
| osteoblasts by direct contact in vitro. | that the cells grew over the entire | that the cells grew over the entire |
| This feature was assessed by staining the | surface of the biomaterial. The | surface of the biomaterial. The |
| cytoskeleton filaments (dye - phalloidin) | number of human osteoblasts growing | number of human osteoblasts growing |
| and cell nuclei (dye - Hoechst33342) 24 | on the curdlan-WPI biomaterial | on the curdlan-WPI-HAp biomaterial |
| and 72 hours after inoculation of human | increased over time. The cells showed | increased over time. The cells showed |
| osteoblasts on biomaterials. | normal morphology. | normal morphology. |

The results presented in Table 1 show that the curdlan-WPI/curdlan-WPI-HAp biomaterial prepared according to the invention shows favorable biological properties in relation to osteoblast cells, i.e., hFOB 1.19 cell line (cells obtained from the American Type Culture Collection (ATCC)). Both the polymer phase and the polymer-ceramic phase of the biomaterial enhance the viability and proliferation of human osteoblasts in vitro, which suggests that the biphasic biomaterial could find potential application in the regeneration of cartilage and bone defects.

1. Biphasic β-1,3-glucan (curdlan) and hydroxyapatite ceramics (HAp)-based biomaterial characterized in that it is composed of β-1,3-glucan (curdlan), whey protein isolate (WPI) and hydroxyapatite ceramics (HAp), where the proportions of polymer components to 100 ml of aqueous solution are, respectively: 6-20% (w/v)—β-1,3-glucan, 20-50% (w/v)—whey protein isolate (WPI), while the amount of added hydroxyapatite ceramic granules (HAp) to such a polymer mixture is 40 g-100 g, whereby the upper phase is a mixture of β-1,3-glucan (curdlan) and whey protein isolate (WPI), and the lower phase is the mixture of β-1,3-glucan (curdlan) and whey protein isolate (WPI) and hydroxyapatite ceramics (HAp).

2. The biomaterial according to claim 1, characterized in that it is sterilized.

3. The biomaterial according to claim 1, characterized in that it contains β-1,3-glucan in an amount of 8% (w/v) with relation to 100 ml of the aqueous solution.

4. The biomaterial according to claim 1, characterized in that the whey protein isolate (WPI) is present in an amount of 30% (w/v) related to 100 ml of the aqueous solution.

5. The biomaterial according to claim 1, characterized in that the hydroxyapatite ceramic (HAp) is in the form of nanopowder or powder or granules measuring 0.01-1.0 mm.

6. The biomaterial according to claim 1, characterized in that the hydroxyapatite ceramic (HAp) is present in an amount of 70-80 g with relation to 100 ml of aqueous polymer solution.

7. A biphasic β-1,3-glucan (curdlan) and hydroxyapatite ceramics (HAp)-based biomaterial described in claim 1 for application in the osseochondral lesions repair.

8. The method for the production of a biphasic curdlan-based biomaterial for the osseochondral lesions repair, characterized in that 20-50% (w/v) aqueous solution of whey protein isolate (WPI), preferably 30% (w/v), is added to the β-1,3-glucan (curdlan) powder so that the concentration of curdlan relative to the WPI solution was 6-20% (w/v), preferably 8% (w/v), then hydroxyapatite ceramic (HAp) in the form of nanopowder or powder or granules of 0.01-1.0 mm is added to such a mixture, baked at a temperature of 500-1300° C., in the amount of 40-100 g per 100 ml of aqueous polymer solution, then the obtained mixture is subjected to centrifugation for 1-10 minutes at a speed of 800-10.000 rpm, then the mixture with formed phases: the upper—polymeric and the lower—polymeric-ceramic, are incubated for preferably 15 minutes at a temperature of 90-120° C.

9. The method according to claim 8, characterized in that the obtained biphasic biomaterial is subjected to sterilization.

10. The method according to claim 8, characterized in that the granules are used in an amount of 40 to 100 g, most preferably 70-80 g (related to 100 ml of an aqueous polymer solution), sintered at a temperature of 1100-1200° C.

11. The method according to claim 9, characterized in that the biphasic biomaterial is sterilized by autoclaving so that it has a wet form.

12. The method according to claim 9, characterized in that sterilization of the wet material is carried out at 121° C. for 15 minutes.

13. The method according to claim 9, characterized in that the biphasic biomaterial is dried at room temperature before gas sterilization.

14. The method according to claim 9, characterized in that gas sterilization of the dry biomaterial is performed with ethylene oxide (55° C.) for up to 3 hours.

15. A method according to claim 8, characterized in that the granule size is 0.05-0.2 mm.

16. The method according to claim 8, characterized in that the obtained mixture is subjected to centrifugation for 3 minutes at a speed of 3000 rpm.

17. The method according to claim 8, characterized in that the mixture with the formed two phases (upper—polymer and lower—polymer-ceramic) is incubated at 90° C.
